(19) United States
(12) Patent Application Publication
(10) Pub. No.: US 2025/0122541 A1
(43) Pub. Date: Apr. 17, 2025

(54) ENZYMATIC AND CHEMICAL CATALYSIS FOR PRODUCTION FROM GLUCOSE OF THE PHARMACEUTICAL PRECURSOR, S-3-HYDROXY- Y -BUTYROLACTONE HBL

(71) Applicants: The United States, as represented by the Secretary of Agriculture, Washington, DC (US); University of Maine, Orono, ME (US); Wisconsin Alumni Research Foundation, Madison, WI (US)

(72) Inventors: Philip J. Kersten, Arena, WI (US); Thomas J. Schwartz, Orono, ME (US); James A. Dumesic, Verona, WI (US)

(21) Appl. No.: 18/913,632

(22) Filed: Oct. 11, 2024

Related U.S. Application Data
(60) Provisional application No. 63/543,588, filed on Oct. 11, 2023.

(51) Int. Cl. C12P 7/62; C12N 9/04; C12N 9/88

(52) U.S. Cl. CPC C12P 7/62; C12N 9/0006; C12N 9/88; C12Y 101/0301; C12Y 402/0111

(57) ABSTRACT

A method of synthesizing(S)-3-hydroxy-γ-butyrolactone (HBL), the method including producing a trione using an enzymatic conversion of glucosone. The enzymatic conversion may include: mutating a domain of Aldos-2-ulose dehydratase (AUDH) to obtain a mutant AUDH; dehydrating glucosone using the mutant AUDH to obtain an intermediate, basidiopyrone P (BPP) (4,5-dihydroxy-2-(hydroxymethyl)-2H-pyran-5-one); and forming the trione from the BPP. The method may further include catalyzing the trione using a base to form an ester of 3,4-dihydroxybutyrate; and converting the ester of 3,4-dihydroxybutyrate into the HBL by adding an acid to the ester of 3,4-dihydroxybutyrate.

### CROSS-REFERENCE TO RELATED APPLICATION

This application is a non-provisional application claiming priority to provisional application No. 63/543,588, filed on Oct. 11, 2023, the entire contents of which are incorporated herein by reference.

### BACKGROUND

### 1. Field

The present disclosure relates to a method of synthesizing a pharmaceutical precursor, more specifically, a method for synthesizing(S)-3-hydroxy-γ-butyrolactone (HBL).

### 2. Description of the Related Art

Americans spend an estimated 550 billion USD on pharmaceuticals each year, amounting to approximately 1200 USD per year in per capita spending. However, this figure is skewed heavily towards those who do not require pharmaceuticals for their daily lives, with some paying upwards of 10,000 USD monthly. Health industry analysts anticipate increased spending as national drug expenditures are forecast to grow to 621 billion USD by 2025. While there is debate as to whether rising drug costs in the U.S. match their production value, these forecasts warrant a deeper look into methods to reduce costs, which are driven largely by materials and production.

One high-cost intermediate is(S)-3-hydroxy-γ-butyrolactone (HBL), a chiral building block used in the synthesis of many pharmaceuticals, including cholesterol-lowering drugs (e.g., Lipitor® and Crestor®), antibiotics (e.g., Linezolid and Ezetimibe), HIV inhibitors, and nutritional supplements, among others in addition to its use as a chemical intermediate when chirality is not important. Presently, nearly all commercially available HBL is derived from petroleum, with wholesale costs reported to be about $450/kg. This high cost is driven by the complex production pipeline for petroleum-derived HBL, wherein maleic anhydride (produced from butane or benzene) is first converted to fumaric acid, which in turn is enzymatically converted to L-malic acid, followed by hydrogenation to produce HBL.

Because of this complex and expensive production pipeline, there have been several attempts to produce HBL sustainably using lower-cost methods. For example, Dhamankar and coworkers used recombinant E. coli that co-overexpresses seven genes to integrate an endogenous glyoxylate shunt with the 3,4-dihydroxybutyric acid (DHB)/HBL pathway. Their optimized cell line achieves 0.3 g L⁻¹ HBL and 0.7 g L⁻¹ DHB (i.e., 24% of the theoretical maximum). While promising, the low titers obtained via this method will likely still result in high costs of HBL due to the challenge of recovering HBL from dilute aqueous solution.

Therefore, there is a need for a simpler and cost-effective method for synthesizing the pharmaceutical precursor, (S)-3-hydroxy-γ-butyrolactone (HBL).

### SUMMARY

HBL production is challenging using approaches featuring solely chemical or solely biological catalysis. Accordingly, one approach combines biological and chemical processing for HBL production. In this regard, the combination of whole-cell enzyme catalysis and chemical catalysis can be particularly fruitful, e.g. for the production of furylglycolic acid (a co-monomer for lactic acid) from glucose. In the present disclosure, the same enzymatic pathway is modified to produce a new, highly reactive intermediate referred to as ‘trione’, which can subsequently be converted to(S)-HBL via homogeneous chemical catalysis. The final HBL product is enantiomerically pure because none of the reactions involve the chiral C5 carbon of glucose. An overview of the sequence of chemical transformations is shown in FIG. 1.

In an embodiment, the present subject matter relates to a method of synthesizing (S)-3-hydroxy-γ-butyrolactone (HBL), wherein the method may include: producing a trione using an enzymatic conversion of glucosone. The enzymatic conversion may include: mutating a domain of Aldos-2-ulose dehydratase (AUDH) to obtain a mutant AUDH; dehydrating glucosone using the mutant AUDH to obtain an intermediate, basidiopyrone P (BPP) (4,5-dihydroxy-2-(hydroxymethyl)-2H-pyran-5-one); and forming the trione from the BPP. The method may further comprise catalyzing the trione using a weak base to form an ester of 3,4-dihydroxybutyrate; and converting the ester of 3,4-dihydroxybutyrate into the HBL by adding an acid to the ester of 3,4-dihydroxybutyrate.

In a further embodiment, the present subject matter relates to a method of synthesizing a pharmaceutical precursor, (S)-3-hydroxy-γ-butyrolactone (HBL), wherein the method may include: producing a trione using an enzymatic conversion of glucose. The enzymatic conversion may include: oxidizing glucose using pyranose-2-oxidase (POX) to obtain glucosone; mutating a domain of Aldos-2-ulose dehydratase (AUDH) to obtain a mutant AUDH; dehydrating the glucosone using the mutant AUDH to obtain an intermediate, basidiopyrone P (BPP) (4,5-dihydroxy-2-(hydroxymethyl)-2H-pyran-5-one); and forming the trione from the BPP. The method may further comprise catalyzing the trione using a weak base to form a mixture of an ester of 3,4-dihydroxybutyrate and glycolate; and converting the ester of 3,4-dihydroxybutyrate and glycolate into the HBL and glycolic acid by adding a strong acid to the mixture.

These and other features of the present subject matter will become readily apparent upon further review of the following specification.

### BRIEF DESCRIPTION OF DRAWINGS

FIG. 1 is a schematic illustrating an overview of the sequence of chemical transformations in an embodiment of a method of synthesizing(S)-3-hydroxy-γ-butyrolactone (HBL).

FIG. 2 is a schematic illustrating the reactions that may be catalyzed by Aldos-2-ulose dehydratase (AUDH) as described herein.

FIG. 3 is an illustration of the structure of Aldos-2-ulose Dehydratase (AUDH).

FIGS. 4A-4D show UV spectral scans with glucosone and cell-free extracts of AUDH variants. FIG. 4A shows wild-type D₁D₂D₃ initially produces BPP which is converted to cortalcerone. FIG. 4B shows the mutation to domain 1 in D₁*D₂D₃ prevents the production of cortalcerone, leading to an accumulation at A265. FIG. 4C shows D₁ (domain 1 alone) has no activity on its own with glucosone. FIG. 4D shows addition of D₁ to a reaction with D₁*D₂D₃ re-establishes wild-type activity as seen with D₁D₂D₃.

FIGS. 5A-5D show graphs demonstrating activity (or lack thereof) of D₁D₂D₃ sonicate with the trione. FIG. 5A shows spectral changes for the positive control of D₁D₂D₃ sonicate with 10 mM glucosone (as in FIG. 4A). FIG. 5B shows the same reaction condition as for FIG. 5A except with 10 mM trione instead of 10 mM glucosone. FIG. 5C shows spectral changes with 10 mM trione alone, i.e., without addition of D₁D₂D₃. FIG. 5D shows differences between FIGS. 5B and 5C.

FIGS. 6A-6B show graphs used for the determination of BPP half-life. FIG. 6A shows normalization of data by setting the maximum observed A265 to 1 and the minimum observed A265 to 0. FIG. 6B shows a plot of the natural log of the normalized data.

FIG. 7 shows graphs providing difference spectra with BPP and APM with D₁. FIG. 7A shows spectral scans of BPP. FIG. 7B shows spectral scans of APM.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

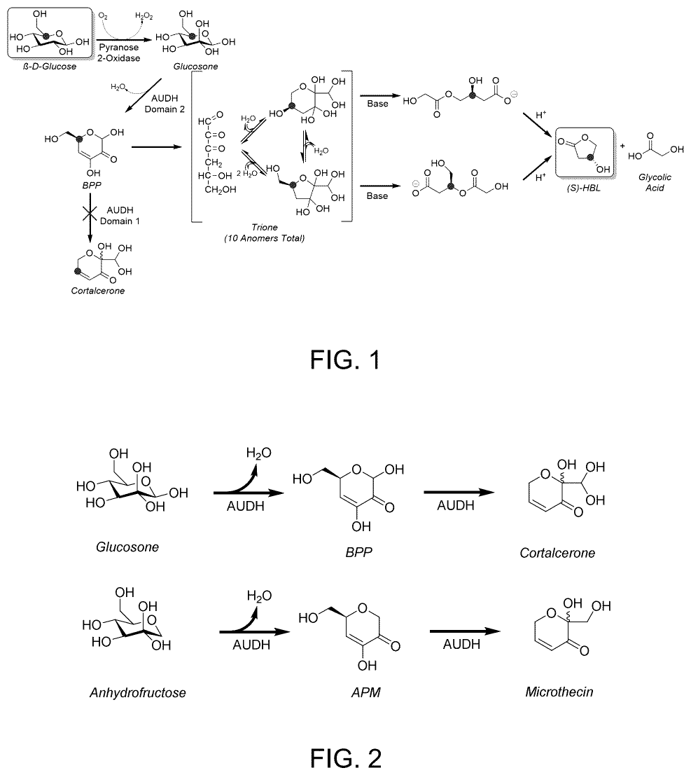

The present subject matter relates to synthesis of HBL. Referring to FIG. 1, the enzymatic and catalytic route from glucose to(S)-3-hydroxy-γ-butyrolactone (HBL) and glycolic acid is illustrated. Pyranose 2-oxidase converts glucose to glucosone, which is then converted to Basidiopyrone P (BPP, IUPAC name 4,5-dihydroxy-2-(hydroxymethyl)-2H-pyran-5-one) via Domain 2 of Aldos-2-ulose dehydratase (AUDH). Domain 1 of AUDH is mutated to inhibit its function, leading to the formation of trione rather than Cortalcerone. Trione undergoes a homogeneously base-catalyzed (e.g., with piperazine) retro-Aldol reaction to form an ester of 3,4-dihydroxybutyrate and glycolate, which is subsequently converted to HBL and glycolic acid upon acidification with HCl. Carbon 5 of glucose indicated by the black dot carried throughout most of the structures in FIG. 1, remains unaffected by any of the catalytic steps, leading to production of chiral(S)-HBL.

The synthetic route to produce trione as shown in FIG. 1 is guided by enzymatic conversion of glucose to cortalcerone, a compound with antimicrobial properties, with some modifications to instead produce the trione. In cortalcerone synthesis, pyranose 2-oxidase (POX) first oxidizes glucose to glucosone with oxygen or quinones as electron acceptors. Aldos-2-ulose dehydratase (AUDH; also known as pyranosone dehydratase) then dehydrates and isomerizes glucosone to cortalcerone. There is a functional connection between POX and AUDH suggested by the location of the genes immediately adjacent to each other in Phanerochaete chrysosporium (protein model 6207208 and 6207226 respectively, https://mycocosm.jgi.doe.gov/Phchr4_2/Phchr4_2.home.html.) This organizational pattern of related genes is also observed in the genomes of other wood-decay fungi.

In an embodiment of the present subject matter, the method may comprise producing a trione using an enzymatic conversion of glucosone. The enzymatic conversion may include mutating a domain of Aldos-2-ulose dehydratase (AUDH) to obtain a mutant AUDH. In various embodiments, the mutant AUDH may have a single amino acid substitution. By non-limiting example, the single amino acid substitution may include substituting Phe for His at position 155. In various embodiments, other amino acids may be used as a substitution for the His at position 155. In some embodiments, the single amino acid substitution may inhibit formation of cortalcerone in the enzymatic conversion of glucose. The method may also include dehydrating glucosone using the mutant AUDH to obtain an intermediate, basidiopyrone P (BPP) (4,5-dihydroxy-2-(hydroxymethyl)-2H-pyran-5-one).

The present methods may also include forming the trione from the BPP and converting the trione using a catalyst to form an ester of 3,4-dihydroxybutyrate. By non-limiting example, weak base catalysts useful in this regard may include piperazine, sodium bicarbonate, tris, bis-tris, or other bases that do not fully dissociate in water. In still other embodiments, a catalyst useful herein may include a Lewis acid. Weak bases as used herein may include a base having a pKa between 6-11. In other embodiments, the weak base may have a pKa of 6.4, 8.1, 9.8, and 10.3, as well as within any ranges having two of these values as endpoints.

The method may further include converting the ester of 3,4-dihydroxybutyrate into the HBL by adding a strong acid to the ester of 3,4-dihydroxybutyrate. By non-limiting example, strong acids useful herein may include hydrochloric acid. In other embodiments, a strong acid useful herein may be any acid that fully dissociates in water. In some embodiments, a strong acid useful herein may include an acid having a pKa<−1 or otherwise allow for reducing pH to between about 2 and about 3. In some embodiments, an amount of acid is added to decrease the pH to between about 2 and about 3. In various embodiments, the strong acid may be added to the ester of 3,4-dihydroxybutyrate to reduce pH to lower than 3. In other embodiments, the strong acid may be added to the ester of 3,4-dihydroxybutyrate to reduce pH to 0.7.

In various embodiments, the intermediate basidiopyrone P (BPP), may have the formula:

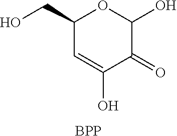 [SMILES: O=C1C(O)=C[C@@H](CO)OC1O ; "BPP"]

In further embodiments, a yield of HBL obtainable from the present methods may be at least 80%, about 80%, or greater than 80%.

In some embodiments, the synthesized HBL may be about 95%, 95%, or greater than 95% chiral(S)-HBL.

In other embodiments, the oxidizing of glucose to glucosone can be performed in an inflatable bag as a reactor. The reaction may occur using a method of repurposing inflatable packaging pillows as bioreactors as described in U.S. patent application Ser. No. 17/237,618, filed Apr. 22, 2021, the disclosure of which is incorporated entirely herein by reference.

By non-limiting example, the trione may be produced by modifying the enzymatic route to obtain cortalcerone. A UV-absorbing intermediate may be observed during catalysis by AUDH with glucosone as a substrate. This may indicate that there are at least two distinct enzymatic steps in the conversion of glucosone to cortalcerone. Additionally, AUDH may also catalyze the conversion of anhydrofructose (AF) to microthecin in an analogous reaction series as illustrated in FIG. 2. In the reaction series illustrated in FIG. 2, an intermediate named ascopyrone M (APM) may be observed. The analogous intermediate observed from glucosone is more reactive and has not previously been isolated. This species is referred to herein by the trivial name basidiopyrone P (BPP; the IUPAC name is 4,5-dihydroxy-2-(hydroxymethyl)-2H-pyran-5-one), consistent with the tradition of naming pyrones from Ascomycetes as ascopyrones (e.g., ascopyrone M from Microthecium; ascopyrone P from Pezizales; ascopyrone T from Tuberales; here basidiopyrone P from Phanerocheate, a basidiomycete). Accordingly, APM and BPP are analogous intermediates in AUDH catalysis with AF and glucosone, respectively (FIG. 2).

Biological catalysis for trione synthesis may be achieved in a two-step process with E. coli whole cells expressing POX for the synthesis of glucosone from glucose and then cells expressing a mutated AUDH (vide infra) that is based on native AUDH and mutated to convert glucosone to trione. In various embodiments, a eukaryotic expression system for producing the POX and AUDH may be used. In one embodiment, glucosone as used herein may be produced from 10 wt % glucose in a weakly buffered solution in overnight incubations. The AUDH variant has a single-amino-acid substitution that prevents the isomerization of BPP that produces cortalcerone (FIG. 1). The design for the AUDH variant is a result of deconvoluting the catalytic roles for the AUDH domains.

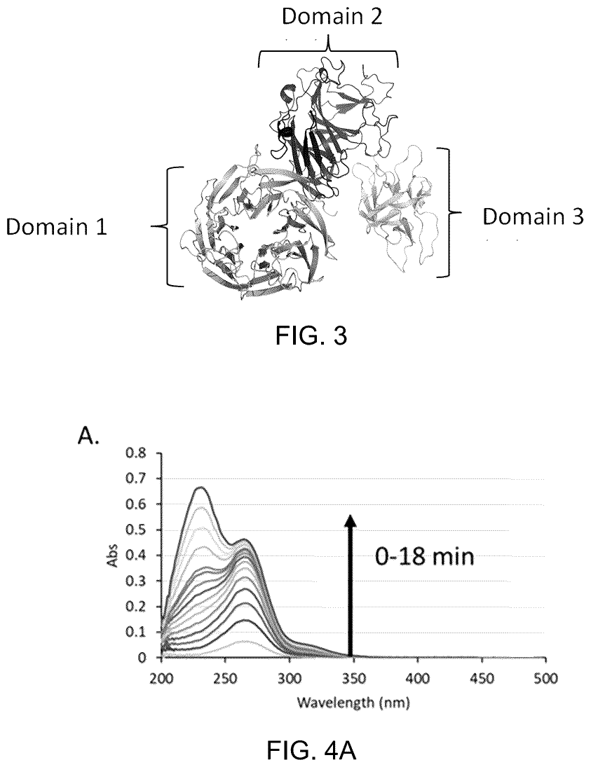

The crystal structure of AUDH, is shown in FIG. 3 in cartoon format. The 900-amino-acid-long enzyme is homo-dimeric and has the following three domains: domain 1 (D₁) consisting of 433 amino acids (residues 1-433) folded into a seven-bladed β propeller, domain 2 (D₂) consisting of 306 amino acids (residues 434-739) with two β-sandwich structures characteristic of a cupin fold, and domain 3 (D₃) with 161 amino acids (residues 740-900) and joined to D₂ by a short linker (FIG. 3). Crystal structure analysis indicates that both D₁ and D₂ complex with the AF reaction intermediate, APM, supporting the conclusion that these two domains are the active sites for catalysis. The C-terminal lectin domain is not likely to be involved in catalysis but may have a role in carbohydrate binding.

AUDH catalyzes two reactions, an initial dehydration of glucosone or AF to produce BPP or APM respectively, and a second isomerization of BPP or APM to cortalcerone or microthecin, respectively. When AUDH is complexed to APM, D₁ may catalyze the initial dehydration both based on the proximity of His155 to the C3 hydroxyl of APM and because of the possibility that the His155 imidazole plays a role in acid/base catalysis. The crystal structure also shows APM hydrogen bonding to His641 in D₂, suggesting that this amino acid may also play some role in catalysis.

To probe the function of D₁ and D₂, a His155Phe (D₁*D₂D₃) was constructed, His641Phe (D₁D₂*D₃), and a truncation containing amino acids 1-433 (D₁) to isolate the activity of each domain. Recombinant expression of all three variants produced a soluble protein as determined by SDS-PAGE analyses. The wild-type D₁D₂D₃ and its variants were tested in whole-cell reactions with glucosone and characterized by NMR at a single time (approximately 18 hours) as shown below in Table 1.

TABLE 1

| Nomenclature | Substrate | Product |
|---|---|---|
| D₁D₂D₃ | Glucosone | Cortalcerone |
| D₁*D₂D₃ | Glucosone | Trione |
| D₁D₂*D₃ | Glucosone | NR |
| D₁*D₂D₃ + D₁D₂*D₃ | Glucosone | Cortalcerone, predominantly |
| D₁ | Glucosone | NR |
| D₁*D₂D₃ + D₁ | Glucosone | Cortalcerone, predominantly |

Table 1: Overnight reactions (18 h) with whole cells and glucosone were evaluated. Activities of domain 1 and domain 2 can be selectively inactivated by single amino acid substitutions and the remaining activities complemented by admixture to reconstitute wild-type activity.
NR, no reaction.

As expected, catalysis with D₁D₂D₃ provided cortalcerone. However, D₁*D₂D₃ produced trione, and there was no reaction of D₁D₂*D₃ with glucosone, but D₁*D₂D₃ and D₁D₂*D₃ complemented to produce cortalcerone. Likewise, the D₁ variant complemented D₁*D₂D₃ to produce cortalcerone, while there was no activity with the D₁ variant alone. This series of reactions provided evidence that D₂ of AUDH catalyzes the initial dehydration of glucosone, and that D₁ is responsible for a subsequent reaction. These results are contrary to previous literature based on crystal structure analysis suggesting that domain 1 is responsible for the initial dehydration of glucosone and that domain 2 catalyzes the isomerization to cortalcerone.

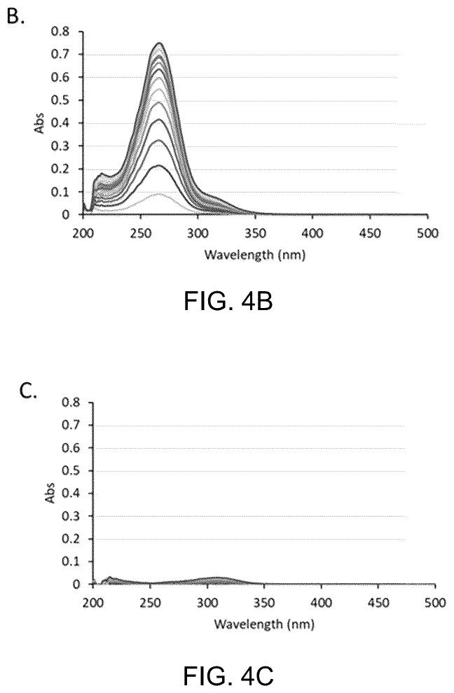

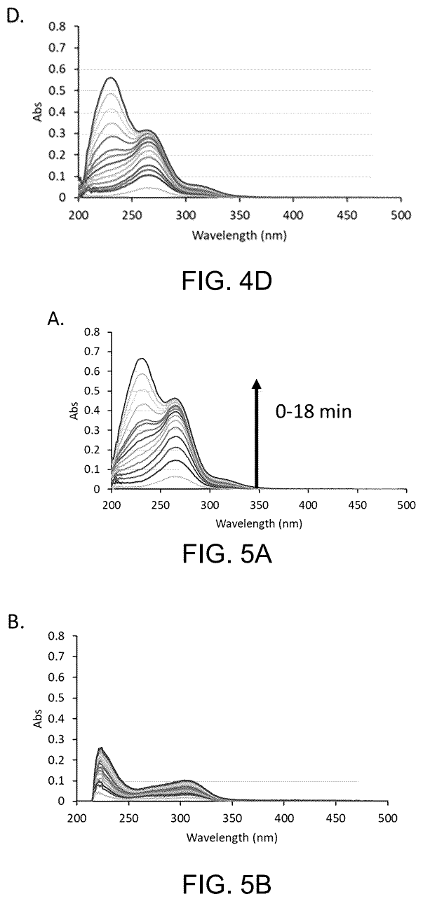

NMR did not provide the analytical sensitivity for detection of low concentrations of possible transient chemical intermediates. For example, BPP was not detected by NMR, though it has been previously identified using UV spectroscopy. Accordingly, the reaction was analyzed via UV spectroscopy using a cell-free extract of AUDH for catalysis, as shown in FIGS. 4A-4D. Wild-type AUDH activity with glucosone leads to an initial accumulation of BPP intermediate (A265) which is followed by cortalcerone formation (A230), consistent with the activity of native AUDH. The D₁*D₂D₃ variant was successful in halting domain 1 activity and consequently showed only BPP production. The D₁ variant showed neither BPP nor cortalcerone production from glucosone; however, a combination of both D₁*D₂D₃ and D₁ re-established wild-type functionality with a similar initial build-up of A265 followed by A230 indicative of cortalcerone production. Collectively, these reactions indicate that catalysis by AUDH is initiated by D₂ with dehydration of glucosone to BPP, followed by D₁ catalysis to produce cortalcerone.

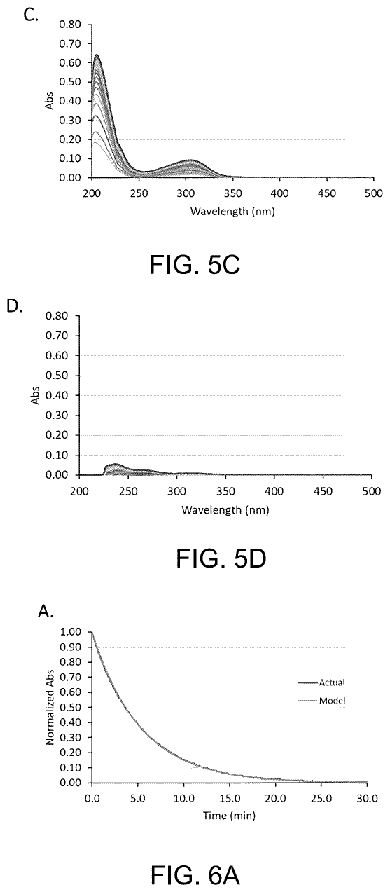

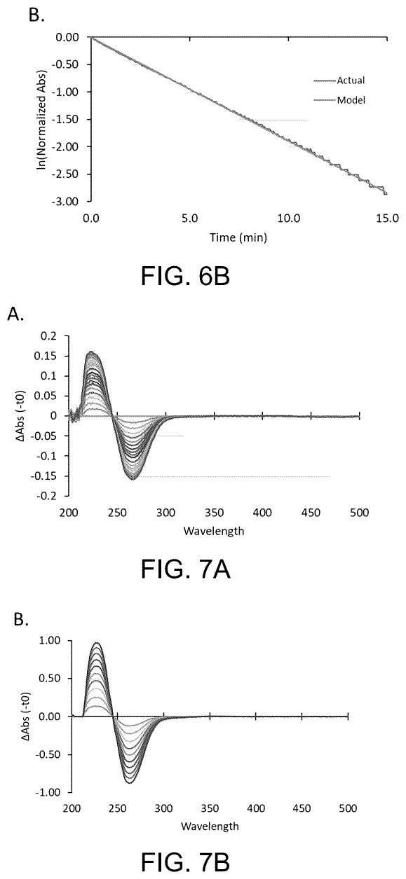

Koths et al. found that BPP is labile and rearranges to a product lacking a UV signature (i.e., the trione as described herein), suggesting that this UV-transparent intermediate is a substrate intermediate for cortalcerone formation by AUDH. See K. Koths, R. Halenbeck, M. Moreland, Synthesis of the antibiotic cortalcerone from D-glucose using pyranose 2-oxidase and a novel fungal enzyme, aldos-2-ulose dehydratase. Carbohydrate Research 232, 59-75 (1992). Importantly, the trione (made by conversion of 555 mM glucosone with D₁*D₂D₃ whole cells) is not a substrate for wild-type D₁D₂D₃ (FIG. 5A-5D), indicating that the trione is a non-enzymatic side-reaction product, not a substrate intermediate for AUDH, contrary to previous interpretations. The ability to accumulate BPP with D₁*D₂D₃ without formation of cortalcerone, then remove D₁*D₂D₃ by ultrafiltration, allowed studies on the stability of the labile intermediate (FIG. 6A-6B). A first-order decay for BPP was observed with a half-life (t_{1/2}) of approximately 3.7 minutes in 0.1 M phosphate, pH 6, the same reaction conditions used by Koths et al.

It was not previously clear whether a single domain or a combination of domains might be responsible for the conversion of BPP to cortalcerone, especially considering that an isomerization could involve hydration-rearrangement-dehydration steps, conceivably achieved by active sites in close proximity. To address this question, BPP was produced with D₁*D₂D₃, the D₁*D₂D₃ was subsequently removed by ultrafiltration, and then spectral changes were analyzed upon addition of D₁. Difference spectra, with the spectral scan zeroed immediately after D₁ addition, show BPP disappearance with the concomitant production of cortalcerone, indicating that only D₁ is required to make cortalcerone from BPP. Furthermore, no intermediate between BPP consumption and cortalcerone production was detected, as demonstrated by the isosbestic point at 245 nm, evidence that the conversion of BPP to cortalcerone is complete before leaving the D₁ active site (FIG. 7A). The analogous series of reactions with AF follows the same path, with first conversion of AF to APM with D₁*D₂D₃ and then conversion of APM to microthecin by D₁ after removal of D₁*D₂D₃ (FIG. 7B).

In an embodiment, the present subject matter relates to a method of synthesizing(S)-3-hydroxy-γ-butyrolactone (HBL). The method may include producing a trione using an enzymatic conversion of glucose. The glucose may have a concentration of 10 wt % in a weakly buffered solution. By way of non-limiting example, the buffer may be a 10 mM citrate having a pH 6 with washed whole cells. In various implementations, the glucose may be β-D-glucose. The enzymatic conversion may include oxidizing glucose using pyranose-2-oxidase (POX) to obtain glucosone.

In various embodiments, by non-limiting example, the POX may be expressed from E. coli whole cells. In some embodiments, the AUDH may be expressed from E. coli whole cells. In another embodiment, another procaryotic expression system may be used. In still other embodiments, a eukaryotic expression system may be used to produce mutant AUDH.

The present synthesis methods may also include mutating domain 1 of Aldos-2-ulose dehydratase (AUDH) to obtain a mutant AUDH. The mutant AUDH may have a Phe in place of the His at position 155 of the 433 amino acids in domain 1. In various embodiments, the His at position 155 may be replaced with other amino acids. In other embodiments, domain 2 of AUDH may be replaced with alternative enzymes having homology to domain 2, including those without a D₁ domain. Such alternative enzymes can be determined by use of, by way of non-limiting example, gene mining databases and the like. The method may also include dehydrating the glucosone using the mutant AUDH to obtain an intermediate, basidiopyrone P (BPP) (4,5-dihydroxy-2-(hydroxymethyl)-2H-pyran-5-one); and forming the trione from the BPP. The method may then include catalyzing the trione using a weak base to form a mixture of an ester of 3,4-dihydroxybutyrate and glycolate; and converting the ester of 3,4-dihydroxybutyrate and glycolate into the HBL and glycolic acid by adding hydrochloric acid to the mixture. In certain embodiments, a “weak base” as described herein may relate to a base that, upon dissolution in water, does not disassociate completely, so that the resulting aqueous solution contain only a small proportion of hydroxide ions, and a large proportion of undissociated molecules of the base. In various embodiments, a weak base may be any base having a pKa of between about 6 and 11, or 6.4, or 8.1, or 9.8, or 10.3. By non-limiting example, a weak base may be selected from the group consisting of piperazine, sodium bicarbonate, tris, and bis-tris. According to this last step, the HBL is the desired product, and the glycolic acid is a by-product.

In various embodiments, the intermediate basidiopyrone P (BPP), may have the formula:

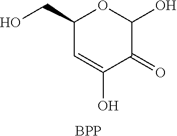 [SMILES: O=C1C(O)=C[C@@H](CO)OC1O ; "BPP"]

In a further embodiment, a yield of synthesized HBL is 90%.

In an embodiment, the synthesized HBL is greater than 99% chiral(S)-HBL.

In another embodiment, the oxidizing of glucose to glucosone is performed in an inflatable bag as a reactor. In various embodiments, the reaction may be performed as described in U.S. patent application Ser. No. 17/237,618, filed Apr. 22, 2021, the entire contents of which are incorporated herein by reference.

One potential application of HBL, which may have high value, is as a pharmaceutical precursor, which depends on its enantiomeric purity. To identify the chirality of the HBL produced by the above process, gas chromatography (GC) with flame ionization detection (FID) was applied using a MilliporeSigma® Supelco® ß-Dex 255 capillary GC column containing a chiral stationary phase of 2,3-diO-acetyl-6-0-TBDMS-ß-cyclodextrin. (MilliporeSigma and Supelco are registered trademarks of Merck KGaA of Darmstadt, Germany) This column successfully separated the enantiomers of HBL, although the retention times depended on the solvent in which the HBL was dissolved. Based on the reaction network from glucose to HBL, the chirality should be preserved at the C-5 position (FIG. 1). The reaction product solution was extracted with ethyl acetate, and the organic phase analyzed by GC/FID. (S)-HBL was observed in this analysis, with no (R)-HBL apparently present, indicating an enantiomeric excess of >99% (S)-HBL. The partition coefficient of HBL extraction into ethyl acetate was 0.1±0.02, and quantification of the HBL in the ethyl acetate coupled with this partition coefficient leads to an overall yield of HBL from glucose of 90%, which is limited by equilibrium, vide supra. HBL can also be extracted into THE, with a partition coefficient of 1.3±0.5, and propylene carbonate, with a partition coefficient of 0.5±0.1.

Extraction and analysis using calculated partition coefficient values of batch syntheses showed a resulting concentration in agreement with prior NMR analysis estimates of 0.14 M. Batch reactions were agitated in the presence of ethyl acetate, and extractions performed using a separatory funnel after 24-hour rest time. In two instances, the rest time was extended to 48 hours and showed an increased concentration of HBL after GC-FID analysis. This result suggests that the equilibrium-limited acidification of DHB shifts towards HBL as it is extracted into the organic phase.

A techno-economic analysis (TEA) was conducted to determine the economic viability of the present synthetic methods, following the discounted cash flow method with an assumed 10% internal rate of return. An annual production capacity of 120 tonnes of HBL was selected based on market demand. A process flow diagram was created to produce HBL with a high purity (>99.9%) and enantiomeric excess (>99%) from glucose. The HBL production process related to 1) POX and AUDH cell synthesis, 2) high purity oxygen production, 3) conversion of sugars (glucose) to glucosone using POX cells, 4) conversion of glucosone to trione using AUDH cells, 5) upgrading trione to DHB using a base catalyst (Piperazine), 6) acidification of DHB to HBL using HCl, and 7) separation and purification of HBL.

In certain embodiments, the air can be compressed to, e.g., 6 bar before being fed to a two-bed pressure swing adsorption (PSA) column with zeolite 13× as adsorbent. The zeolite 13× in the PSA column can adsorb nitrogen and produces oxygen with a purity greater than 95%. Two seed fermentation trains, each consisting of five seed reactors, can be used to produce the initial and make up POX and AUDH cells.

In an embodiment, hanging bag reactors (developed by Leidos® of Reston, Virgina) are supplied with oxygen and POX cells, along with glucose, catalase, sodium citrate, and water. In various embodiments, a recombinant catalase may be used for whole cell protection from peroxide. Within these reactors, the POX cells can facilitate the conversion of glucose to glucosone. The POX cells, which can be separated from the broth containing water and glucosone using a centrifuge, can be recycled back to the hanging bag reactor. In a similar manner, a second train of hanging bag reactors can be employed to produce trione from glucosone using AUDH cells.

In certain embodiments, continuous stirred tank reactors can be chosen for the formation of DHB and GA from the trione in the presence of a base catalyst, and for the subsequent acidification of DHB salt using HCl to produce HBL. The product stream can contain HBL, GA, water, and a range of impurities. In certain embodiments, the product stream is sent to the first extraction column, where approximately 50 wt % of HBL is extracted into ethyl acetate. The raffinate from the first extraction column is routed to the second extraction column to extract remaining HBL to ethyl acetate. Finally, a distillation column and vacuum evaporation unit are employed to recover ethyl acetate and produce HBL at a purity higher than 99.9%. The glycolic acid (GA) byproduct can typically end in the wastewater along with other impurities, although the GA can also be recovered and purified for sale as a co-product.

In certain embodiments, ASPEN plus software can be used to size process equipment for the separation and purification train, continuous stirred reactor, and the compressor. The remaining units can be sized using rules of thumb (seed trains and centrifuge), vendor data (hanging bag reactors), and published simulation data (PSA).

In conducting the TEA analysis as described above, the process equipment size was selected as a basis to assess the installed equipment costs, which were then updated to 2022 dollars using chemical engineering cost indices. Finally, the cost factors developed by the National Renewable Energy Laboratory (NREL) as a function of the total installed equipment cost were used to determine the additional costs, such as site development and project contingency, associated with the total capital investment. The material and energy balances were used to calculate the operating costs. The capital and the annual operating costs to produce HBL at the selected scale were determined to be $32.6 MM and $15.6 MM, respectively. Approximately 40% of the operating costs are attributed to the cost of piperazine. At this scale, the TEA analysis predicts a minimum selling price of HBL of $162 per kg, which is 64% lower than the current state-of-the-art.

The aforementioned pathway to the production of enantiomerically pure HBL may not only be more sustainable than existing methods (e.g., using biomass feedstocks, mild temperatures, and aqueous solvent), but it is also more financially favorable. Moreover, while not explored in depth in the economic analysis, the methods described herein yield GA as a byproduct, which can be used in the formation of various plastics, a topic of increasing importance which has spurred research interest in its synthesis from biomass sugars. Industrial scale production of GA is generally delivered as technical grade, in aqueous solvent, and so sale as a co-product would require little additional investment beyond what is outlined above for HBL. In contrast, metabolic engineering approaches have been used to produce glycolate from glucose, although such methods typically rely on monocultured, engineered organisms and are generally not tolerant of feedstock impurities and require expensive preprocessing of real biomass feeds. Should the methods described herein be implemented, the anticipated reduction in production costs could have numerous downstream benefits for drug manufacturers and ultimately for consumers.

The combination of biological and chemical catalysis presented here for synthesis of HBL may provide advantages not realized by using metabolic-pathway engineering alone. Instead of catalyzing a biological pathway from glucose to HBL, a simpler pathway from glucose to trione was developed, requiring fewer biocatalysts and producing a small, neutral product, i.e., the trione. This approach may allow a two-step process (glucose to glucosone, and glucosone to trione) using whole cells where membrane permeability is not an issue and cell viability is not required. This approach may also circumvent the need for fed-batch cultures (to prevent toxic conditions), co-feedstock additions (to compensate for metabolic deficiencies) and pH control (to neutralize the organic acid product). Furthermore, the steps for the synthesis of the trione can be optimized individually for best characteristics and for high expression. An important enabling discovery for this approach is the synthesis of new enzymes with homology to D₂ that can replace D₁*D₂D₃ in the synthesis of the trione by whole-cell catalysis. As indicated previously, such new enzymes with homology to D₂ can be obtained by certain known methods, such as, by way of non-limiting example, gene database mining. Such alternative enzymes include but are not limited to those having no activity with anhydrofructose and glucosone, those having activity with just anhydrofructose, and those having activity with both anhydrofructose and glucosone. These alternatives with activity with glucosone can be much smaller (239 to 362 amino acids) than the 900 amino acid sequence of AUDH. Further, such alternative enzymes can, but not necessarily, be expressed in E. coli for whole-cell catalysis.

The approach as described herein for synthesis of HBL via a trione intermediate has a broad range of applications. By way of non-limiting example, xylose is a substrate for POX for production of xylosone which is a substrate for AUDH; the analogous reaction series with the resulting xylo-trione would provide 3-hydroxy propionic acid (HPA) and glycolic acid. HPA is one of the bio-based platform chemicals identified by DOE with greatest potential for commercial applications. The economic viability for HPA from xylose would not be expected to be as favorable as(S)-HBL from glucose because xylose is a poorer substrate than glucose for POX, and the price of HPA is much lower than chiral(S)-HBL.

The present teachings can be illustrated by the following examples.

### EXAMPLES

### Materials and Methods

Bacterial strains and plasmids. The cDNA sequence (GenBank accession number AY522922) encoding POX of P. chrysosporium BKMF-1767 (ATCC 24725) with an N-terminal T7-tag and a C-terminal His6 tag, was synthesized for optimized expression in E. coli, inserted in pJ414 (ATUM, Newark, CA, USA) and transformed into BL21 (DE3). The cDNA sequence (GenBank accession number KF699142) encoding AUDH from P. chrysosporium RP78, a homokaryotic strain derived from BKM-F-1767, was synthesized with a C-terminal His6 tag for optimized expression in E. coli, inserted in pJ414 (ATUM, Newark, CA, USA) and transformed into BL21 (DE3). This wild-type AUDH with C-terminal tag is designated D₁D₂D₃ (see Table 2). The AUDH variants are based on D₁D₂D₃ sequence with amino acid substitutions and deletions achieved by DNA synthesis (ATUM, Newark, CA, USA). All constructs are transformed in E. coli BL21 (DE3). The structure of AUDH is understood in the art as provided in Crystal Structure of Bifunctional Aldos-2-Ulose Dehydratase/Isomerase from Phanerochaete chrysosporium with the Reaction Intermediate Ascopyrone M. Claesson, et al., Crystal Structure of Bifunctional Aldos-2-Ulose Dehydratase/Isomerase from Phanerochaete chrysosporium with the Reaction Intermediate Ascopyrone M, J. Molecular Bio., Vol. 417, Iss. 4, April 2012, pp. 279-293, the entirety of which is incorporated herein by reference.

TABLE 2

| Nomenclature | Protein Structure |
|---|---|
| D₁D₂D₃ | Wild-type AUDH GenBank accession |
| | KF699142 plus C-terminal |
| | GSGSHHHHHH tag. |
| D₁*D₂D₃ | His155Phe variant of D₁D₂D₃. |
| D₁D₂*D₃ | His641Phe variant of D₁D₂D₃. |
| D₁ | N-terminal domain (residues 1-433) plus |
| | a C-terminal GSGSHHHHHH tag. |

Protein Modelling. The PyMol model for AUDH is based on PDB entry 4A7K using cDNA sequence (GenBank accession number KF699142) encoding AUDH from P. chrysosporium RP78, a homokaryotic strain derived from BKM-F-1767. This 900-amino-acid sequence differs at 4 positions from the AUDH of P. chrysosporium ATCC 32629 on which the PDB entry is based.

Growth and preparation of cells for catalysis. E. coli cells for all studies reported here (e.g., in glucosone synthesis, trione synthesis, and AUDH variant characterizations) were prepared as follows. Auto-induction medium with modifications were used. Briefly, each liter of auto-induction medium contains 48 g terrific broth (TB) powder, 2 mM MgSO₄, 0.2× trace metals (1000× trace metals, Teknova Cat. No: T1001), 28 mM di-sodium succinate, 0.005% antifoam 204, 8 g glycerol, 0.15 g glucose, and 5 g lactose. Carbenicillin (100 μg ml⁻¹ final) provides selection. 500 mL of inoculated auto-induction medium in 2-L baffled flasks are incubated at 250 rpm, 20° C. for 30 h. Cells are harvested as 40-ml aliquots in 50-ml conical centrifuge tubes at 4000×g for 15 min. The supernatants are removed, and the cell pellets overlaid with 10 mM citrate pH 6, 0.9% saline buffer to prevent freezer burn before storage at −20° C. Prior to use of the cells in catalysis, the frozen cells are thawed and washed twice (40 ml) in 0.9% saline, 10 mM pH6 citrate buffer.

### Example 1

### Synthesis of Glucosone

Synthesis of glucosone. Glucosone was prepared from 0.55M glucose (10% w/v) in 10 mM citrate having pH 6 with washed whole cells in disposable pillow bioreactors. Cells expressing POX from 40 mL of culture (about 2 g wet cells) were sufficient to catalyze a 200-mL reaction in 18 h. Catalase from bovine liver (Sigma C40) at 10 mg/200 mL reaction provided protection from peroxide.

### Example 2

### Synthesis of Trione

Trione was prepared from 40 mL of 0.55M glucosone (adjustment to pH 6, as necessary) in a 50-mL conical centrifuge tube containing of D₁*D₂D₃ washed cells (derived from 40 mL of culture, about 2 g wet weight cells) with mixing end-over-end (20 rpm) overnight (about 18 h). The trione solution was recovered by centrifugation and then sterilized by ultrafiltration (Steriflip®, 0.22 μm Millipore®). (SteriflipÂ® and MilliporeÂ® are registered trademarks of Merck KGaA of Darmstadt, Germany.)

### Example 3

AUDH Variant Characterization with Whole Cells by NMR

Reactions used 1.5 mL of 0.55M glucosone in a 2-mL Eppendorf® tube together with washed cell pellets (as described previously) derived from 1.5 mL of wild-type or AUDH variant cultures. (Eppendorf is a registered trademark of Eppendorf of Hamburg, Germany) Mixing was end-over-end (20 rpm) for approximately 18 h and the supernatant recovered for analysis after centrifugation.

### Example 4

AUDH Variant Characterization with Cell-Free Extract by UV-Spectroscopy

UV-spectroscopic analyses required a homogenous enzyme solution. Cells derived from 40 mL of culture (as described herein) were sonicated (Qsonica Model Q700) in 10 mL of sonication buffer (50 mM Tris pH 8, 50 mM NaCl, 5% glycerol), centrifuged, and aliquots of 1.5 mL were stored at −20° C. Spectroscopic activity was measured using a microplate reader (BioTek Epoch 2). Quartz cuvette scans were taken from 200-500 nm in 1 nm steps using the sweep read mode for a total read time of 15 s. Reactions were in 0.1 M phosphate buffer, pH 6 and contained 10 mM glucosone and 5 μL of sonicate to a final volume of 500 μL. Spectra were processed by subtracting the first spectra (t=0) to remove background noise and allow easier interpretation of results. To test activity with trione as substrate, 10 mM trione was used instead of 10 mM glucosone.

As illustrated in FIGS. 4A-4D show UV spectral scans with glucosone and cell-free extracts of AUDH variants. FIG. 4A shows wild-type D₁D₂D₃ initially produces BPP (λl max=265 nm) which is converted to cortalcerone (λmax=230 nm). FIG. 4B shows the mutation to domain 1 in D₁*D₂D₃ prevents the production of cortalcerone, leading to an accumulation at A265. FIG. 4C shows D₁ (domain 1 alone) has no activity on its own with glucosone. FIG. 4D shows addition of D₁ to a reaction with D₁*D₂D₃ re-establishes wild-type activity as seen with D₁D₂D₃. Collectively, these reactions indicate that catalysis is initiated by domain 2, and domain 1 then catalyzes the subsequent reaction to cortalcerone from an intermediate generated by domain 2.

FIGS. 5A-5D show graphs demonstrating activity (or lack thereof) of D₁D₂D₃ sonicate with the trione. FIG. 5A shows spectral changes for the positive control of D₁D₂D₃ sonicate with 10 mM glucosone (as in FIG. 4 panel A). FIG. 5B shows the same reaction condition as for FIG. 5A except with 10 mM trione instead of 10 mM glucosone. FIG. 5C shows spectral changes with 10 mM trione alone, i.e., without addition of D₁D₂D₃, indicating some non-negligible signal drift from the trione. FIG. 5D shows differences between FIGS. 5B and 5C, indicating a nearly identical drift with or without enzyme present in the reaction.

### Example 5

### Half-Life of BPP

The synthesis and t_{1/2} of BPP was determined spectroscopically at 265 nm. The synthesis of BPP with D₁*D₂D₃ sonicate and 10 mM glucosone in a 500 μL reaction was followed until the absorbance reached ˜1.4 (about 14 minutes). The reaction was then passed through a 3,000 MWCO Nanosep centrifugal filter (PALL Life Sciences) by centrifuging for 4 minutes at 14,000×g and 200 μL of filtrate followed at A265 every 4 s. The decay of A265 was normalized to the highest and lowest measured values, with the highest A265 set to 1 and the lowest to 0. To determine whether the t_{1/2} was 0^{th}, 1^{st} or 2^{nd} order, the natural log of the data was plotted against time. After determining that the decay followed 1^{st}-order decay, t_{1/2} was calculated using the Solver add-in for Microsoft Excel, minimizing the sum of squared differences between the measured and modeled data calculated using Equation 1.

$$Normalized Abs = 1 * 0 . {5}^{t / {t}_{1 / 2}} \qquad ( Equation 1 )$$

FIGS. 6A-6B show graphs used to determine BPP half-life. BPP from glucosone and D₁*D₂D₃ sonicate was monitored at 265 nm after ultrafiltration to remove the protein catalyzing its formation. FIG. 5A shows normalization of data by setting the maximum observed A265 to 1 and the minimum observed A265 to 0. FIG. 5B shows the natural log of the normalized data plotted, and the resulting fit to a straight line indicates the intermediate has a 1st-order decay with a half-life (t_{1/2}) of 3.7 minutes in 0.1 M phosphate, pH 6.

### Example 6

D₁ Activity with BPP and APM

BPP was generated from glucosone as described for the t_{1/2} measurements and APM was generated similarly using AF (Carbosynth Ltd). Briefly, reactions with D₁*D₂D₃ sonicate and glucosone/AF were tracked, and 500 μL of the reaction passed through a 3,000 MWCO Nanosep centrifugal device (PALL Life Sciences) by centrifuging at 14,000×g for 4 minutes. Domain 1 activity was determined as described for AUDH variant characterizations by UV spectroscopy, except for the substrate being replaced by filtrate containing BPP or APM.

FIG. 7 shows graphs providing difference spectra with BPP and APM with D₁. FIG. 7A shows spectral scans of BPP. FIG. 7B shows spectral scans of APM.

### Example 7

### Trione Upgrading

Into the trione solution obtained from whole-cell catalysis a weak base catalyst, in this instance piperazine, was dissolved, to achieve a base concentration of 0.3M. The base converted the trione via retro-aldol reaction to a glycolate ester (GE), which in turn underwent hydrolysis to yield equal parts glycolic acid (GA) and 3,4-dihydroxy butyric acid (DHB). Quantitative in situ NMR spectroscopy revealed the reaction achieved ca. 95% yield (with the other 5% forming polyols) in 1 hour and at ambient temperature. The final stage of the process involved the addition of a homogeneous acid, in this instance HCl, to reduce the pH to 0.7 and facilitate lactonization of DHB. The acidification reaction achieved 88% conversion after 1 hour at ambient temperature.

Chiral GCFID analysis of the final HBL product suggests the product is enantiomerically purse S-HBL. ¹³C NMR analysis of pure S-HBL and the synthesized HBL show corresponding peaks. The final HBL product can be recovered from aqueous solution by liquid-liquid extraction into ethyl acetate (Kp=0.1) or THF (Kp=1.3).

Verification of the chirality of the resulting HBL was done using gas chromatography flame ionization detection (GCFID). For GC experiments, the MilliporeSigma® Supeco® β-Dex 225 Capillary GC chiral column was used with helium carrier gas, an inlet temperature of 250° C., a column temperature of 205° C., a FID temperature of 300° C., and a flow of 1 mL/min.

NMR was conducted on a Varian INOVA 400 spectrometer with D₂O added for signal lock, with a ¹³C frequency of 100.569 MHz. Spectra were acquired by coaveraging 2000 transients with a 5.750 us pulse width and a 1.303 s acquisition time.

It is to be understood that the method of production for pharmaceutical precursor, (S)-3-hydroxy-γ-butyrolactone (HBL) is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A method of synthesizing(S)-3-hydroxy-γ-butyrolactone (HBL), the method comprising:
producing a trione using an enzymatic conversion of glucosone;
wherein the enzymatic conversion comprises:
mutating a domain of Aldos-2-ulose dehydratase (AUDH) to obtain a mutant AUDH;
dehydrating glucosone using the mutant AUDH to obtain an intermediate, basidiopyrone P (BPP) (4,5-dihydroxy-2-(hydroxymethyl)-2H-pyran-5-one); and
forming the trione from the BPP;
catalyzing the trione using a base to form an ester or salt of 3,4-dihydroxybutyrate; and
converting the ester or salt of 3,4-dihydroxybutyrate into the HBL by adding an acid to the ester of 3,4-dihydroxybutyrate.

2. The method of claim 1, wherein the acid is added to the ester of 3,4-dihydroxybutyrate to reduce pH to lower than 3.

3. The method of claim 1, wherein an amount of acid is added to decrease the pH to between about 2 and about 3.

4. The method of claim 1, wherein the mutant AUDH has a single amino acid substitution.

5. The method of claim 4, wherein the single amino acid substitution inhibits formation of cortalcerone.

6. The method of claim 1, wherein the intermediate basidiopyrone P (BPP), has the formula:

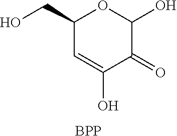 [SMILES: O=C1C(O)=C[C@@H](CO)OC1O ; "BPP"]

7. The method of claim 1, wherein the base is a weak base having a pKa of between 6-11.

8. The method of claim 1, wherein a yield of HBL is greater than 80%.

9. The method of claim 1, wherein the synthesized HBL is greater than 95% chiral(S)-HBL.

10. The method of claim 1, wherein the oxidizing of glucose to glucosone is performed in an inflatable bag as a reactor.

11. The method of claim 1, wherein the mutated domain of AUDH is domain 1.

12. A method of synthesizing(S)-3-hydroxy-γ-butyrolactone (HBL), the method comprising:
producing a trione using an enzymatic conversion of glucose;
wherein the enzymatic conversion comprises:
oxidizing glucose using pyranose-2-oxidase (POX) to obtain glucosone;
mutating a domain of Aldos-2-ulose dehydratase (AUDH) to obtain a mutant AUDH;
dehydrating the glucosone using the mutant AUDH to obtain an intermediate, basidiopyrone P (BPP) (4,5-dihydroxy-2-(hydroxymethyl)-2H-pyran-5-one); and
forming the trione from the BPP;
catalyzing the trione using a base to form a mixture of an ester of 3,4-dihydroxybutyrate and glycolate; and
converting the ester of 3,4-dihydroxybutyrate and glycolate into the HBL and glycolic acid by adding an acid to the mixture.

13. The method of claim 12, wherein the glucose has a concentration of about 20 wt % or less in a weakly buffered solution.

14. The method of claim 12, wherein the glucose is β-D-glucose.

15. The method of claim 12, wherein the POX is combined with an electron receptor selected from the group consisting of oxygen and quinones to obtain the glucosone.

16. The method of claim 12, wherein the intermediate basidiopyrone P (BPP), has the formula:

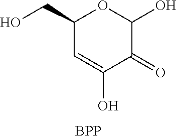 [SMILES: O=C1C(O)=C[C@@H](CO)OC1O ; "BPP"]

17. The method of claim 12, wherein the domain of the AUDH is mutated by a single-amino-acid substitution that prevents the isomerization of BPP to form cortalcerone.

18. The method of claim 12, wherein a yield of synthesized HBL is greater than 80%.

19. The method of claim 12, wherein the synthesized HBL is 95% or higher chiral(S)-HBL.

20. The method of claim 12, wherein the oxidizing of glucose to glucosone is performed in an inflatable bag as a reactor.
